(19) United States
(12) Patent Application Publication
(10) Pub. No.: US 2023/0118746 A1
(43) Pub. Date: Apr. 20, 2023

(54) MEDICAL APPARATUS, METHOD FOR RECORDING A MODEL DATASET, DATA PROCESSING PROGRAM, AND PROGRAM MEMORY MEDIUM

(71) Applicant: Aesculap AG, Tuttlingen (DE)

(72) Inventors: Georg Hettich, Freiburg (DE); Tobias Martin, Obernheim (DE); Allan Maas, Konstanz (DE)

(21) Appl. No.: 18/081,452

(22) Filed: Dec. 14, 2022

Related U.S. Application Data
(63) Continuation of application No. PCT/EP2021/066213, filed on Jun. 16, 2021.

(30) Foreign Application Priority Data
Jun. 18, 2020 (DE) 10 2020 116 073.9

(51) Int. Cl. A61B 34/10; A61B 34/20

(52) U.S. Cl. CPC A61B 34/10; A61B 34/20; A61B 2034/102; A61B 2034/105; A61B 2034/2057

(57) ABSTRACT

A medical apparatus, medical method, data processing program and program storage medium. A piece of reference position information of a reference element arranged on a bone is checked for agreement with a position of the reference element in a reference coordinate system, which is defined by a marking device and can be monitored by a navigation device. The model data set can be registered in the reference coordinate system.

### CROSS-REFERENCE TO RELATED APPLICATIONS

This application is a continuation of International Application No. PCT/EP2021/066213 filed on Jun. 16, 2021 and claims the benefit of German Application No. 10 2020 116 073.9 filed on Jun. 18, 2020. The contents of International Application No. PCT/EP2021/066213 and German Application No. 10 2020 116 073.9 are incorporated herein by reference in their entireties and for all purposes.

### FIELD

The present disclosure relates to medical apparatuses generally, and more specifically to a medical apparatus, comprising a navigation device for detecting at least one medical marking device, a data processing device for processing position data of the navigation device, wherein a position of the at least one marking device is determinable, and a storage device in which a model data set with a spatial representation of a bone is storable. The present disclosure also relates to a method for registering a model data set. Furthermore, the present disclosure relates to a data processing program and a program storage medium.

### BACKGROUND

An exemplary possible application of the present disclosure is, for example, in a surgical procedure regarding defects of the bone, which bone may be, e.g., the human pelvis (pelvic bone). Therefore, as an example, the use of the apparatus and the method in revision surgeries in hip endoprosthetics can be explained in the following, though the disclosure is not limited to this application and is suitable for use outside of revision surgeries. In the latter case, in particular, there are bad conditions from the beginning for the presence and/or substance or quality of the bone. In the typical case, the revision of a hip implant, in particular the replacement of an artificial acetabulum, in the presence of higher-grade bone defects is still a great challenge for a surgeon. For example, when bone substance is lacking or is damaged in the region of the acetabulum, the surgeon lacks an orientation aid intraoperatively for positioning the revision implant. The surgeon can be assisted in the revision procedure with the help of comparisons of the abnormal side of the pelvic bone with the healthy side of the pelvic bone, for example X-ray images of the pelvic bone being used. However, in practice it occurs that even experienced surgeons lack the necessary orientation due to the lack of anatomical landmarks on the pelvic bone, thus negatively affecting the implantation outcome. The surgeon's comprehensive experience in revision surgeries is always helpful and advantageous. A navigation device can assist the surgeon in the orientation and positioning of the revision implant.

For planning the procedure, in particular a revision surgery, for example, a model data set of the pelvic bone can be created. It is desirable to bring the model data set into agreement with the real pelvic bone with assistance from the navigation device. However, the precision of the navigation greatly depends on the precision of the referencing of the pelvic bone by way of the surgical marking device. This can prove to be inadequate.

To adapt model data sets to the real pelvic bone, it is known to preoperatively and intraoperatively record characteristic landmarks of the pelvic bone. Serving as a reference is, e.g., the anterior pelvic plane (APP), by way of which a sort the body's own coordinate system is defined. However, this requires additional expenditure in data recording and processing.

DE 10 2018 116 558 A1 describes a medical instrumentation and a medical method in which a planning data set is created for the surgeon on the basis of an actual state data set of a bone that is considered to be defective. The planning data set may be used intraoperatively for improving the surgery outcome.

US 2018/0116728 A1 describes an apparatus and a method for performing a surgical procedure in which instructions regarding surgical technique are provided to the surgeon by way of a head-mounted display.

### SUMMARY

In a first aspect of the disclosure, a medical apparatus is provided. The medical apparatus comprises:
a navigation device for detecting at least one medical marking device,
a data processing device for processing position data of the navigation device, wherein a position of the at least one marking device is determinable,
and a storage device in which a model data set with spatial representation of a bone is storable, in particular of a pelvic bone, wherein the model data set comprises a piece of characteristic information characterizing a piece of reference position information of a reference element arranged in a defined spatial position on the bone.

The data processing device is configured and programmed to provide a reference coordinate system on the basis of position data of a first marking device arranged in a defined spatial position relative to the bone.

The data processing device is configured and programmed to determine a piece of characteristic information characterizing the position of the reference element in the reference coordinate system on the basis of position data obtained by means of a further marking device.

The data processing device is configured and programmed to bring the position of the reference element in the reference coordinate system into agreement with the reference position information in the model data set.

The data processing device is configured and programmed to, based on the agreement, register the model data set in the reference coordinate system with regard to an overlap of the model data set and the bone.

In a second aspect of the disclosure, a medical method for registering a model data set is provided. The medical method comprises:
providing a model data set with a spatial representation of a bone, wherein the model data set comprises a piece of characteristic information characterizing a piece of reference position information of a reference element arranged on the bone in a defined spatial position;
providing a reference coordinate system on the bone on the basis of position data of a first marking device arranged in a defined spatial position relative to the bone;
determining a piece of characteristic information characterizing the position of the reference element in the reference coordinate system on the basis of position data obtained by means of a further marking device;
providing an agreement of the position of the reference element in the reference coordinate system with the reference position information in the model data set; and
registering, based on the agreement, the model data set in the reference coordinate system with regard to an overlap of the model data set and the bone.

In a third aspect of the disclosure a data processing program is provided with program code means for use in surgery supported by means of a data processing device. The data processing program, when it is running on the data processing device or is loaded onto the data processing device, prompts the data processing device to perform the method in accordance with the second aspect of the disclosure.

In a fourth aspect of the disclosure a program storage medium is provided. The program storage medium comprises the data processing program in accordance with the third aspect of the disclosure. The program storage medium may be non-volatile or volatile.

### BRIEF DESCRIPTION OF THE DRAWINGS

The foregoing summary and the following description may be better understood in conjunction with the drawing figures, of which:

FIG. 1 shows a perspective schematic depiction of a medical apparatus in accordance with the disclosure for performing the method in accordance with the disclosure, wherein a navigation device and a data processing device as well as a surgeon with a display device, and a patient undergoing a revision surgery are depicted;

FIG. 2 shows schematically a pelvic bone of the patient, from which a model data set is created by means of the X-ray radiation;

FIG. 3 shows the physical bone of the patient with a surgical marking device attached thereto, as well as a palpating tool for palpating a reference element; and

FIG. 4 shows the model data set according to FIG. 2, which is depicted superimposed over the bone according to FIG. 3 using the marking device.

### DETAILED DESCRIPTION

Although the disclosure contains illustrations and descriptions of specific embodiments, the disclosure is not intended to be limited to the details shown. Rather, various modifications may be made in the details without departing from the scope of the disclosure.

The present disclosure relates to a medical apparatus, comprising:
a navigation device for detecting at least one medical marking device,
a data processing device for processing position data of the navigation device, wherein a position of the at least one marking device is determinable,
and a storage device in which a model data set with spatial representation of a bone is storable, in particular of a pelvic bone, wherein the model data set comprises a piece of characteristic information characterizing a piece of reference position information of a reference element arranged in a defined spatial position on the bone,
wherein the data processing device is configured and programmed:
to provide a reference coordinate system on the basis of position data of a first marking device arranged in a defined spatial position relative to the bone,
to determine a piece of characteristic information characterizing the position of the reference element in the reference coordinate system on the basis of position data obtained by means of a further marking device,
to bring the position of the reference element in the reference coordinate system into agreement with the reference position information in the model data set, and
to, based on the agreement, register the model data set in the reference coordinate system with regard to an overlap of the model data set and the bone.

In the present disclosure, a reference element arranged in a defined spatial position on the bone is contained in a model data set having the bone in a defined spatial representation.

For this purpose, a piece of characteristic information characterizing the reference position information may be contained in the model data set. The model data set may comprise or form a sort of base coordinate system of the bone itself. For example, the bone can be referenced intraoperatively by way of a (first) marking device, which is detectable by means of the navigation device, for defining the reference coordinate system. A piece of characteristic information of the physical reference element can be obtained by means of a further marking device. The characteristic information can be provided by detecting the further marking device in the reference coordinate system and thus be determined characterizing the position of the reference element in the reference coordinate system. Based thereon, the data processing device can bring the position of the reference element in the reference coordinate system into agreement with the reference position information in the model data set on the basis of the two pieces of characteristic information, because it is known that the characteristic information obtained on the physical reference element has a counterpart in the model data set, namely the characteristic information contained therein. This enables the data processing device to register the model data set in the reference coordinate system when there is agreement. In this way, the base coordinate system of the bone from the model data set can transferred into the reference coordinate system, and information contained in the model data set that relate to the spatial representation of the bone are known in this reference coordinate system. The model data set can thus be “pinned” to the physical bone in a sense and can be tracked by way of the navigation device and the data processing device.

Flowing into the present disclosure is the thought that the position of the reference element defined relative to the bone can be used by a comparison of the characteristic information in the model data set, on the one hand, and physically by way of the reference coordinate system, one the other hand, being compared with one another. In contrast to conventional apparatuses, one is not reliant on a complex and insufficient registration of a model data set on the actual anatomy of the bone, which there can prove to be problematic in particular in the case of bone defects. Furthermore, a referencing of the bone by way of auxiliarly obtained additional information, for example the locations of the anterior pelvic plane in the model data set and intraoperatively, can be omitted.

“Position” in the present case may be understood to mean, in particular, the “location and/or orientation” in the respective coordinate system.

Preferably an implant on the bone may be used as a reference element. The use of the apparatus proves to be advantageous in particular in a revision surgery in which the implant is repositioned or exchanged for another implant.

The registration of the model data set in the reference coordinate system offers the possibility, in particular, of displaying information contained in the model data set on a display device, in particular a spatial representation of the bone. By way of the marking device, it is possible, in particular, to pin this depiction visually in the field of view of a surgeon, which will be explained in the following.

The navigation device may, for example, be of optical configuration and comprise a stereo camera or a mono camera and marker elements. Here, for example, passive markers (in particular retroreflective markers), active (non-emissive), and/or AR markers (augmented reality) may be used. Marker elements may be spatially in non-variable relationship to one another, for example as a so-called “rigid body”, and/or be freely positionable relative to one another.

Provision may be made that the apparatus comprises an X-ray device and that the model data set is creatable using the X-ray device. For example, a computer tomograph is used to create a CT image of the bone including the reference element in order to provide the model data set.

The model data set favorably comprises a three-dimensional representation of the bone.

Beyond the spatial representation of the bone, the model data set may advantageously comprise further information that may be advantageous for performing the procedure.

For example, the model data set comprises information relating to a removal of an implant.

For example, the model data set comprises information relating to an implantation of an implant.

For example, the model data set comprises information relating to a surgical procedure.

For example, the model data set comprises information relating to a surgical technique.

Information of the kind described above can be output, for example, to output planning information and intraoperatively to output instructions to the surgeon for performing the procedure. Information of that kind can be displayed, for example, on the aforementioned display device

As already mentioned, the reference element may preferably be an implant. The use of an implant anchored in the bone as a reference element for providing the reference position information on the one hand and the position in the reference coordinate system on the other hand proves to be reliable and simple. For example, the patient undergoes a revision surgery with the implant being reoriented or exchanged. In this case, the surgeon must surgically expose the implant, such that the surgeon provides access to the implant as a reference element during the procedure. In this way, it is easily possible for the surgeon to obtain the characteristic information intraoperatively on the implant. As a result of the fixed spatial arrangement of the implant in the model data set on the one hand and physically on the bone on the other hand, a respective exact reference is provided, which enables a registration of the model data set and the base coordinate system in the reference coordinate system in a very reliable manner.

The implant may be, e.g., a hip implant, in particular an artificial acetabulum, or a knee implant.

The characteristic information of the reference element may be expressed in different ways.

In an advantageous embodiment, the characteristic information characterizing the reference position information is or comprises a characteristic geometric structure of the reference element. If, for example, the model data set is created by means of CT, a characteristic geometric structure of an implant used as a reference element can emerge therein and be reliably used to define the reference position information.

For example, the characteristic geometric structure is a surface portion of the reference element or comprises such a surface portion. If, for example, an artificial hip socket is used as a reference element, the surface portion may be, for example, a rim of the hip socket that can be recognized intraoperatively by the surgeon when the access is opened.

Therefore, it is advantageous to use as a characteristic geometric structure a surface portion that can be reached by the surgeon with little effort for determining the position of the reference element in the reference coordinate system.

It may be advantageous, in particular, if the data processing device is configured and programmed to determine an agreement of the reference position information with the position of the reference element by comparing the geometric structure with a geometric comparison structure, which comparison structure is determinable on the basis of position data that are determinable using the further marking device. Here, the geometric structure in the model data set can be compared with the intraoperatively detectable comparison structure. This enables the data processing device to determine the agreement. In particular, a surface portion of the reference element and, in particular, implant in the model data set can be compared with a surface portion that is determined by means of the further marking device.

The apparatus favorably comprises a surgical palpating tool on which the further marking device is arranged and with which a surface of the reference element can be palpated for determining the position of the reference element in the reference coordinate system. The palpating tool has, for example, a palpating tip with which the reference element can be palpated. The marking device can be detected by the navigation device, for example automatically. Alternatively, provision may be made that the apparatus comprises an input element, for example a hand or foot switch, after the actuation of which points on the reference element referenced by the palpating tool are registered.

Provision may be made, for example, that the palpating tool is movable along a predetermined or predeterminable path on a surface of the reference element in order to determine the position of the reference element. In the case of an artificial hip socket, for example, a rim of the hip socket is palpated with the palpating tool. This movement is detected by way of the navigation device, and the data processing device can determine the shape of the rim and compare same with the corresponding geometric structure in the model data set.

Alternatively or in addition, provision may be made that the position of the reference element is determinable on the basis of a plurality of characteristic points on a surface of the reference element, which can be palpated by means of the palpating tool. For example, it may be possible to palpate a plurality of points on the rim of the hip socket. Here, it is preferably already possible using thee points to determine a plane defined by the rim, and to determine the shape of the rim by way of the distance of the points from one another.

Provision may be made that instructions for guiding the palpating tool are provided to the surgeon, for example on a display device of the apparatus.

The data processing device is preferably configured and programmed to determine the characteristic information on the basis of an analysis of the model data set. For example, CT data can be automatically analyzed by the data processing device by means of algorithms executable thereon in order to identify the reference element and to extract the characteristic information. Here, in particular, they may be image processing algorithms.

Alternatively or in addition, provision may be made that the characteristic information in the model data set is effected at least partially with intervention by a surgeon.

The data processing device is preferably configured and programmed to determine the characteristic information on the basis of an already known type of the reference element. For example, the type of the implant may be identifiable or predeterminable. For example, a characteristic geometric structure in the model data set can be determined on the basis of the type.

It is favorable if the data processing device is configured and programmed to determine the characteristic information on the basis of an individualized reference element, in particular a patient-specific implant. For example, patient-related information regarding the reference element may be present, on the basis of which the characteristic information can be unambiguously determined.

As already mentioned, it is favorable if the apparatus comprises at least one display device. The model data set is preferably at least partially depictable on the at least one display device. Here, in particular, a spatial and preferably three-dimensional representation of the bone can be displayed on the display device.

The display device may, for example, be stationary. Alternatively or in addition, provision may be made that a display device is arranged on a portable data processing device, in particular a tablet computer (presently including a smartphone or the like).

A display device can preferably be arranged on a head-mounted device that can be worn by a surgeon on their head. This may be data glasses, for example, which are worn by the surgeon such that the image content with the model data set can be displayed in the field of view of the surgeon.

The use of the head-mounted device enables the surgeon, for example, to look at the physical bone and to display the image information of the model data set in the sense of augmented reality (AR).

If a different kind of display device is used, the same effect can be achieved, for example, by an image of the physical bone being taken by means of a camera and information from the model data set being displayed superimposed on said image displayed on the display device.

The data processing device is preferably configured and programmed to determine the position of the display device in the reference coordinate system. For this purpose, for example, provision may be made that the display device is detectable by the navigation device. To this end, for example, a marking device may be arranged on the display device.

In dependence on the determined position of the display device in the reference coordinate system, the model data set can preferably be displayed superimposed over the bone by the data processing device by means of augmented reality. The model data set can be optically “pinned” to the bone in a sense and is thereby preferably in the field of view of the surgeon when they look at the bone.

For example, instructions relating to a removal of an implant can be displayed on the display device.

For example, instructions relating to an implantation of an implant can be displayed on the display device.

For example, instructions relating to a surgical procedure can be displayed on the display device.

For example, instructions relating to a surgical technique can be displayed on the display device.

Provision may be made that the marking device is attachable to the bone in a direct invasive manner, for example with a bone screw, or in an indirect non-invasive manner. A marking may be attachable to the bone in a non-invasive manner, for example by means of a bandage.

As mentioned at the outset, the present disclosure also relates to a method.

Further, the disclosure relates to a medical method for registering a model data set, comprising:
providing a model data set with a spatial representation of a bone, wherein the model data set comprises a piece of characteristic information characterizing a piece of reference position information of a reference element arranged on the bone in a defined spatial position;
providing a reference coordinate system on the bone on the basis of position data of a first marking device arranged in a defined spatial position relative to the bone;
determining a piece of characteristic information characterizing the position of the reference element in the reference coordinate system on the basis of position data obtained by means of a further marking device;
providing an agreement of the position of the reference element in the reference coordinate system with the reference position information in the model data set; and
registering, based on the agreement, the model data set in the reference coordinate system with regard to an overlap of the model data set and the bone.

The advantages that can be achieved with the method in accordance with the disclosure have already been explained in the context of the description of the apparatus in accordance with the disclosure. Reference may be made to the preceding remarks in this regard.

Advantageous embodiments of the method result from advantageous embodiments of the apparatus in accordance with the disclosure. To avoid repetition, reference may be made to the preceding remarks in this regard.

The present disclosure also relates to a data processing program. A data processing program in accordance with the disclosure comprises program code means for use in surgery supported by means of a data processing device, wherein the data processing program, when it is running on the data processing device or is loaded onto the data processing device, prompts the data processing device to execute the medical method described above.

Furthermore, the present disclosure relates to a program storage medium, comprising the data processing program of the kind described above. The program storage medium may be non-volatile or volatile.

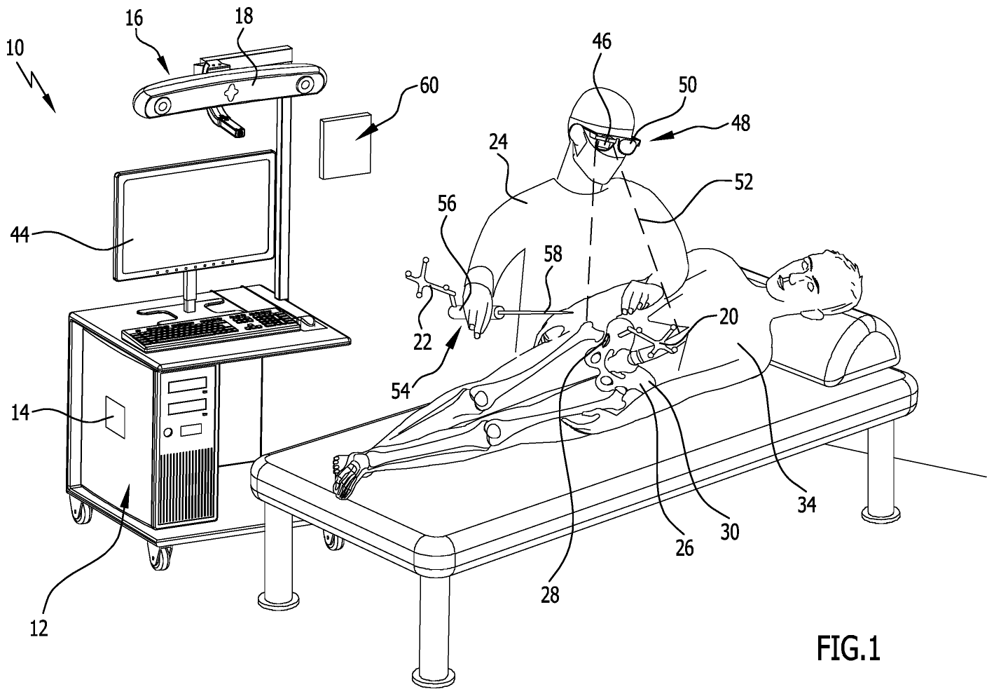

FIG. 1 shows in a perspective depiction an advantageous embodiment of a medical apparatus in accordance with the disclosure, denoted with the reference numeral 10. The apparatus 10 comprises a data processing device 12, for example configured as a computer with a microprocessor and volatile or non-volatile memory. A data processing program in accordance with the disclosure can run on the data processing device 12 and can be provided, for example, with a program storage medium in accordance with the disclosure. The apparatus 10 further has a storage device 14, which presently is integrated into the data processing device 12.

Furthermore, the apparatus 10 comprises a navigation device 16. In the present embodiment, the navigation device 16 is of optical configuration and comprises a camera, in particular a stereo camera 18. With the stereo camera 18, radiation, in particular infrared radiation that is emitted by a marking device 20 of the apparatus 10, can be detected.

The marking device 20 and a further marking device 22 of the apparatus 10 are presently of passive configuration. Radiation emitted by the stereo camera 18 is reflected by the marking devices 20, 22 and is received again in the stereo camera 18. Alternatively or in addition, the use of active marking devices 20, 22 is possible, which emit radiation on their part.

In the present embodiment, the marking devices 20, 22 are configured as rigid bodies (also referred to as “navigation stars”), which are known per se and have marker elements in a defined spatial arrangement to one another.

The navigation device 16 forms an optical measuring system, which can determine position data of the marking devices 20, 22. This makes it possible to track the marking devices 20, 22 in order to determine their location and their orientation as a function of time. Position data of the navigation device 16 can be provided to the data processing device 12 for a corresponding analysis.

Alternatively or in addition to the optical navigation device 16 described here, a different kind of navigation device may be provided.

The data processing device 12 may be a constituent part of the navigation device 16, or vice versa.

In the present case, the apparatus 10 is used to assist the surgeon 24 in a revision surgery. The present example concerns the removal of an implant 28 implanted in a bone 26.

In the described example, the bone 26 is the pelvic bone 30 and the implant 28 is an artificial hip socket 32 that was implanted in the patient 34 in a previous procedure.

The hip socket 32 is inserted into the acetabulum 36 and has a bowl-shaped portion 38, which engages into the acetabulum 36, and a rim 40. The rim 40 is arranged on a surface 42 of the hip socket 32.

The apparatus 10 may comprise at least one display device. Presently, a stationary display device 44 in the form of a monitor, which is known per se, is provided. Moreover, the apparatus 10 comprises a display device 46.

The display device 46 is arranged on a head-mounted device 48, configured as data glasses 50. The data glasses 50 can be worn by the surgeon 24 like conventional glasses. Content can be displayed in the field of view 52 of the surgeon 24 by way of the display device 46, said content being preserved when the head of a surgeon 24 moves.

The display devices 44, 46 are controllable by the data processing device 12 to display the image contents.

The head-mounted device 48 is presently preferably provided with a marking device (not shown). This makes it possible to detect the device 48 by means of the navigation device 16. A movement of the device 48 in space can be tracked. This makes it possible to determine the location and orientation of the field of view 52 in space.

In the present case, the marking device 20 forms a reference coordinate system. The position of the marking device 22 (location and orientation) can be determined in the reference coordinate system. Correspondingly, the position of the device 48 in the reference coordinate system can be determined.

The apparatus 10 comprises a surgical palpating tool 54. The surgeon 24 can palpate characteristic points with the palpating tool 54. The palpating tool 54 comprises a grip element 56 and a palpating tip 58 arranged thereon. The marking device 22 is fixed to the palpating tool 54.

Due to the defined spatial relationship of the marking device 22 to the palpating tip 58, the data processing device 12 can determine which points in the reference coordinate system are palpated.

The apparatus 10 further comprises an X-ray device 60 that is schematically depicted in FIG. 1. The X-ray device 60 is, in particular, a CT device for creating CT images of the bone 26.

The apparatus 10 can be used in revision surgery for the hip socket 32, for example, as follows, in the context of an advantageous embodiment of the method in accordance with the disclosure:

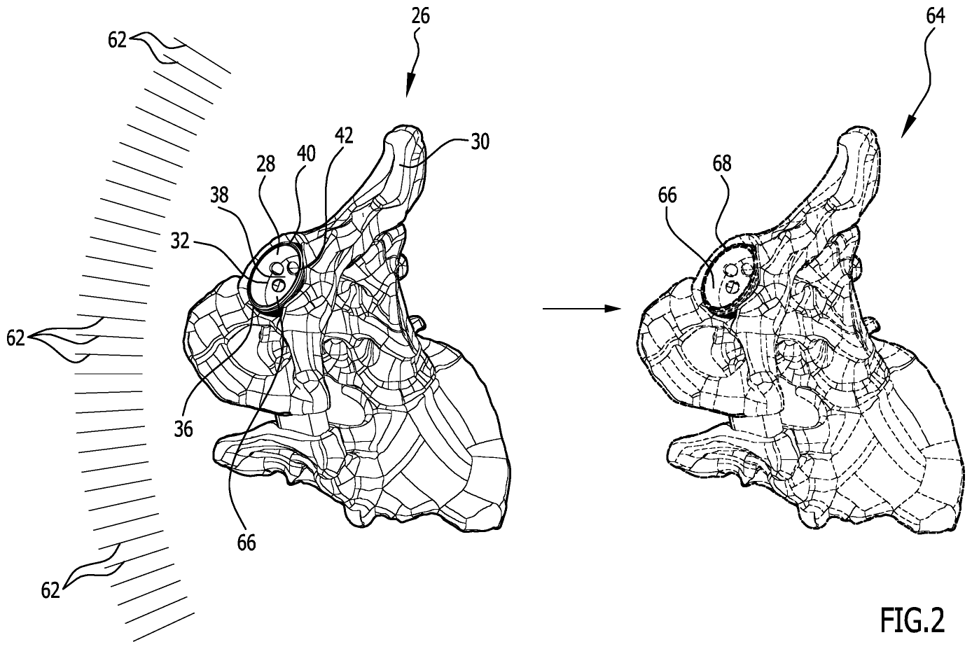

Preoperatively, a model data set 64 of the pelvic bone 30 can be created to assist in the navigated procedure. For this purpose, a model data set 64 can be created by means of the X-ray device 60, the radiation 62 of which is schematically depicted in FIG. 2. The model data set 64 comprises a spatial representation of the pelvic bone 30. Said representation is, in particular, presently a three-dimensional representation.

The hip socket 32 is clearly apparent in the model data set 64. Because the position of the hip socket 32 on the pelvic bone 30 is exactly defined, the hip socket 32 can be used as a reference element 66 for the registration of the model data set 64 in the reference coordinate system, which will be explained in the following. The hip socket 32 defines a piece of characteristic information, which is characteristic for the reference position information of the hip socket 32 on the pelvic bone 30.

In the present example, in particular, a characteristic structure 68 is used as characteristic information. In the present example, the rim 40 of the hip socket 32 can be used for the characteristic structure 68. However, this is not limiting for the present disclosure.

It is thus possible in the model data set 64 to extrapolate the position of the hip socket 32 as a reference element 66 in the pelvic bone 30 from the characteristic structure 68.

Here, further information known about the implant 28 can be taken into account, for example the type of implant or the fact that it is a patient-specific implant 28 with a known structure.

FIG. 2 shows schematically the pelvic bone 30 subject to the radiation 62 and an exemplary representation of the model data set 64 on which the characteristic structure 68 is highlighted.

The model data set 64 can be stored in the storage device 14. In addition to the spatial representation of the pelvic bone 30, further information may be contained in the model data set 64, for example relating to a removal of the implant 28, relating to an implantation of an implant, for example repositioning of the implant 28 or insertion of a new implant, relating to a surgical procedure, and/or relating to a surgical technique.

Intraoperatively, the surgeon 24 can attach the marking device 20 to the pelvic bone 30 for defining the reference coordinate system. A patient-stationary reference is thus formed.

When the access is opened and the hip socket 32 is exposed, in particular its rim 40 can be identified and accessed by the surgeon 24 in a simple manner.

Just like the characteristic structure 68 in the model data set 64 has a defined spatial position, the hip socket 32, in particular the rim thereof 40, has a defined spatial position on the actual pelvic bone 30. This makes it possible to compare the rim 40 for agreement with the characteristic structure 68.

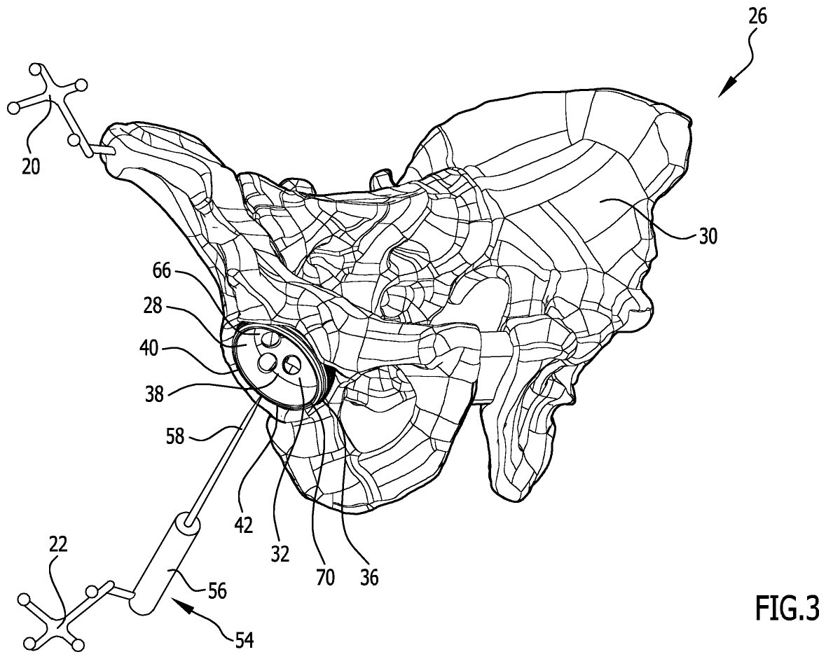

For this purpose, the surgeon 24 can palpate the rim 40 by means of the palpating tool 54. This movement can be detected by the navigation device 16 and be evaluated by the data processing device 12, the position data in the reference coordinate system being determined (FIG. 3).

The rim 40 forms a characteristic comparison structure and therefore a piece of characteristic information that characterizes the position of the implant 28 as a reference element 66 in the reference coordinate system.

The position of the implant 28 in the reference coordinate system can be brought into agreement with the reference position information in the model data set 64. For this purpose, the characteristic structure 68 can be compared with and checked for agreement with the characteristic comparison structure 70.

When there is agreement, it is possible, in particular, to register the model data set 64 in the reference coordinate system. Because the reference position information in the model data set 64 can be applied to the pelvic bone 30 on the basis of the characteristic comparison structure 70, the data processing device 12 can associate the position of the characteristic comparison structure 70 in the reference coordinate system with the position of the characteristic structure 68 in the model data set 64.

The model data set 64 is thereby pinned to the physical pelvic bone 30 in a sense. When the pelvic bone 30 moves in space, for example as the result of the patient 34 being repositioned, the model data set 64 can be tracked.

The model data set 64 can be displayed on the display devices 44, 46. For example, a depiction on the display device 46 is advantageous.

Because the position of the device 48 in the reference coordinate system is known, the display content of the display device 46 can be displayed such that the representation of the pelvic bone 30 is depicted at the correct position by means of augmented reality (AR). Because the pelvic bone 30 itself can only be partially seen due to the limited access, it cannot be seen as a whole by the surgeon 24. The system makes it possible by pinning the model data set 64, for example, to make the complete three-dimensional representation of the pelvic bone 30 visible at the correct position in the field of view 52.

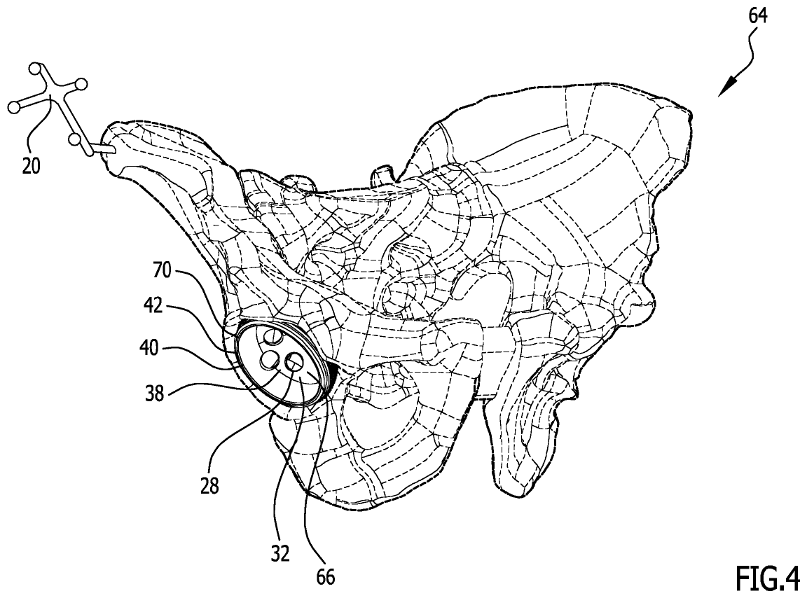

This is depicted as an example in FIG. 4, intending to symbolize that the view of the physical hip socket 32 is possible, as well as of the marking device 20, whereas the structure of the pelvic bone 30 is derived from the model data set 64 (dashed lines).

The apparatus 10 in accordance with the disclosure and the method in accordance with the disclosure make it possible to use the geometrically defined structures of the implant 28.

The use of anatomic structures, in particular with high-grade bone defects that make a referencing of the model data set 64 more difficult, is not necessary.

Moreover, it is not necessary to bring characteristic anatomic points of the pelvic bone 30 in the model data set 64 and intraoperatively into agreement with one another. However, this can still be advantageous for increasing precision. For example, the surgeon 24 can use the palpating tool 64 to palpate characteristic points like, e.g., the spina iliaca anterior superior.

During the procedure, possible information for instructing the surgeon 24 can be displayed on the display device 46. Conceivable here is, in particular, information for removing the implant 28, for inserting a new implant, or for repositioning the implant 28, information relating to the surgical procedure, and/or the surgical technique.

1. A medical apparatus comprising:
a navigation device for detecting at least one medical marking device;
a data processing device for processing position data of the navigation device, wherein a position of the at least one medical marking device is determinable; and
a storage device in which a model data set with spatial representation of a bone is storable, wherein the model data set comprises a piece of characteristic information characterizing a piece of reference position information of a reference element arranged in a defined spatial position on the bone,
wherein the reference element is an implant, and
wherein the data processing device is configured and programmed to:
provide a reference coordinate system based on position data of a first marking device of the at least one medical marking device, the first marking device arranged in a first defined spatial position relative to the bone,
determine a piece of characteristic information characterizing a position of the reference element in the reference coordinate system based on position data obtained by a second marking device of the at least one medical marking device,
bring the position of the reference element in the reference coordinate system into agreement with the reference position information in the model data set, and
register the model data set in the reference coordinate system with regard to an overlap of the model data set and the bone.

2. The medical apparatus according to claim 1, further comprising an X-ray device, wherein the model data set is creatable using the X-ray device.

3. The medical apparatus according to claim 1, wherein the model data set comprises a three-dimensional representation of the bone.

4. The medical apparatus according to claim 1, wherein the model data set comprises at least one of:
information relating to a removal of the implant;
information relating to an implantation of the implant;
information relating to a surgical procedure; and
information relating to a surgical technique.

5. The medical apparatus according to claim 1, wherein the implant is a hip implant or a knee implant.

6. The medical apparatus according to claim 1, wherein the characteristic information characterizing the reference position information is or comprises a characteristic geometric structure of the reference element.

7. The medical apparatus according to claim 6, wherein the characteristic geometric structure is or comprises a surface portion of the reference element.

8. The medical apparatus according to claim 6, wherein the data processing device is configured and programmed to determine an agreement of the reference position information with the position of the reference element by comparing the geometric structure with a characteristic geometric comparison structure that is determinable based on position data that are determinable using the second marking device of the at least one medical marking device.

9. The medical apparatus according to claim 1, further comprising a surgical palpating tool,
wherein the second marking device of the at least one medical marking device is arrangeable on the surgical palpating tool, and
wherein a surface of the reference element is configured to be palpated with the surgical palpating tool for determining the position of the reference element in the reference coordinate system.

10. The medical apparatus according to claim 9, wherein the surgical palpating tool is movable along a predetermined or predeterminable path on the surface of the reference element to determine the position of the reference element.

11. The medical apparatus according to claim 9, wherein the position of the reference element is determinable based on a plurality of characteristic points on the surface of the reference element, the plurality of characteristic points configured to be palpated by the surgical palpating tool.

12. The medical apparatus according to claim 1, wherein the data processing device is configured and programmed to at least one of:
a) determine the characteristic information based on an analysis of the model data set,
b) determine the characteristic information based on an already known type of the reference element, and
c) determine the characteristic information based on an individualized reference element.

13. The medical apparatus according to claim 1, further comprising at least one display device, wherein the model data set is, at least partially, displayable on the at least one display device.

14. The medical apparatus according to claim 13, wherein the at least one display device is at least one of:
stationary; and
arranged on one of:
the data processing device, which comprises a portable data processing device, and
a head-mounted device.

15. The medical apparatus according to claim 13, wherein the data processing device is configured and programmed to determine a position of the at least one display device in the reference coordinate system and to, in dependence thereon, display the model data set superimposed over the bone by augmented reality.

16. The medical apparatus according to claim 13, wherein the at least one display device is configured to display instructions relating to at least one of the following:
instructions relating to a removal of the implant,
instructions relating to an implantation of the implant,
instructions relating to a surgical procedure, and
instructions relating to a surgical technique.

17. The medical apparatus according to claim 1, wherein the at least one medical marking device is attachable to the bone in a direct invasive manner or in an indirect non-invasive manner.

18. A medical method for registering a model data set comprising the steps of:
providing a model data set with a spatial representation of a bone, wherein the model data set comprises a piece of characteristic information characterizing a piece of reference position information of a reference element arranged on the bone in a defined spatial position;
providing a reference coordinate system on the bone based on position data of a first marking device arranged in a defined spatial position relative to the bone;
determining a piece of characteristic information characterizing the position of the reference element in the reference coordinate system based on position data obtained by a second marking device;
providing an agreement of the position of the reference element in the reference coordinate system with the reference position information in the model data set; and
registering, based on the agreement, the model data set in the reference coordinate system with regard to an overlap of the model data set and the bone.

19. A data processing program with program code for use in surgery supported by a data processing device, wherein the data processing program is configured to prompt the data processing device to perform the method according to claim 18 when the data processing program is running on the data processing device or is loaded onto the data processing device.

20. A program storage medium, comprising the data processing program according to claim 19.
